(19) United States
(12) Patent Application Publication
(10) Pub. No.: US 2010/0197978 A1
(43) Pub. Date: Aug. 5, 2010

(54) IPA From Propylene Using Mixed Metal Oxides

(76) Inventors: Jihad M. Dakka, Whitehouse Station, NJ (US); Doron Levin, Highland Park, NJ (US); Stephen McCarthy, Center Valley, PA (US); Travis Allen Reine, Overijse (BE); Mary Jo Schneider, Baton Rouge, LA (US); James C. Vartuli, Bradenton, FL (US); Charles M. Yarbrough, Baton Rouge, LA (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 12/598,009

(22) PCT Filed: Apr. 10, 2008
(86) PCT No.: PCT/US08/59881

§ 371 Date: Mar. 17, 2010

Related U.S. Application Data
(60) Provisional application No. 60/931,423, filed on May 23, 2007.

(51) Int. Cl. C07C 29/04; B01J 23/10; B01J 21/06

(52) U.S. Cl. 568/900; 502/304; 502/308

(57) ABSTRACT

The invention relates to the production of isopropyl alcohol (IPA) by direct hydration of propylene over mixed transition metal oxides co-precipitated with ZrO₂. In embodiments the mixed metal oxides have improved hydrolytic stability and are active over a wider temperature range than existing direct hydration catalysts.

### FIELD OF THE INVENTION

The invention relates to the production of isopropyl alcohol (IPA) by direct hydration of propylene over mixed metal oxides.

### BACKGROUND OF THE INVENTION

Hydration of propylene to make isopropyl alcohol (IPA) has been practiced commercially for many years. Commercial processes may be classified as indirect or direct hydration processes. Indirect hydration processes contact propylene (C₃═) with strong mineral acids such a sulfuric acid or phosphoric acid to form a sulfate/phosphate ester which is then hydrolyzed to reform the acid and produce the IPA product. Direct hydration (DH) processes feed propylene and water over a solid acid catalyst that hydrolyzes the olefin to produce IPA.

DH processes typically use solid acid catalyst or heterogeneous catalyst for the hydration of the propylene directly to IPA. In the direct process, the hydration of the olefins to alcohols is carried out directly and in a single step, by contacting the olefin with the hydration water in the presence of an acidic catalyst. DH processes typically require chemical grade propylene or better as feed to decrease impurities produced and maintain catalyst life. DH may be carried out in vapor-phase, liquid-phase or mixed phase. IPE is the major by-product from the C₃⁼ direct hydration processes.

Solid acid catalysts that have been utilized for DH include functionalized divinylbenzene polymer catalysts such as sulfonic acid resins (i.e. Amberlysts™, Dowexs™), solid phosphoric acid (SPA), various types of zeolites, metal impregnated silica and aluminas. Although these catalysts are capable of DH chemistry they all are subject to various limitations that impact how they can be used. Resin catalysts are very active but are not intrinsically selective and chemically degrade over 150° C. To manage the non-selectivity, high water to C3=ratios are used to drive the equilibrium towards IPA. These high water systems create more costly processes for separation and purification of IPA. Many zeolites/molecular sieves and other silica/alumina catalysts are intrinsically selective to IPA due to microporous structure, but these have hydrolytic stability problems.

Accordingly, this very important commercial process is an area of active research to overcome the problems with prior art processes.

One specific area of research involves catalysts based on the modification one metal oxide by incorporation of other metal oxides, generally referred to as mixed metal oxides (MMO's). Typically, at least one of the metals is a transition metal (i.e., Groups 3-11 according to the Periodic Table from Chemical and Engineering News, 63(5), 27, 1985). As used herein, the term “transition metal” includes the members of the Lanthanide and Actinide families of said Table.

Work by Hoecker in 1996 (DE 1012783) identified ZrO₂ for use as a substrate for a phosphoric acid coated catalyst. The activity from that catalyst is believed due to the inclusion of phosphoric acid.

Krause (WO 200234390) described catalysts comprised silica plus 5-50% oxides of the transition metals, including ZrO₂. These mixed metal oxides are based primarily on the silica substrate.

Chiyoda Corp (JP 08224472) reported a water insoluble zirconium hydroxide coated with a tungsten compound.

See also recent work directed to IPA production as disclosed in U.S. Pat. No. 7,173,158; U.S. Patent Application Publication No. 20060269462 and 20060270883; and PCT publication WO 2006/135475.

In previous work described by one or more of the present inventors, a CeO/ZrO₂ mixed metal oxide catalyst was identified as active for the decomposition of isopropyl ether (IPE). See U.S. Pat. No. 7,102,037, and U.S. Application Publication No. 2006-0281954. High purity C₃═ was produced, along with some isopropyl alcohol (IPA) was produced as a side reaction. Since these reactions are reversible, either IPE or IPA can be optimized as the main product by selection of the reaction conditions.

While it has been found that the CeO/ZrO₂ catalyst previously described promotes direct hydration of propylene, the activity is insufficient for commercial production.

The present inventors have found, however, that by proper incorporation of certain other metal oxides by the use of co-precipitation, an MMO catalysts based on ZrO₂ for the direct hydration of propylene may be achieved.

### SUMMARY OF THE INVENTION

The invention is directed to mixed metal oxide (MMO) catalysts based on ZrO₂. The mixed metal oxide comprises at least two metals, at least one of which is zirconium (Zr) and having at least one other metal selected from transition metals other than Zr. The mixed metal oxides of the invention are made by co-precipitation of the at least two metals. The mixed metal oxides according to the invention are useful for the production of isopropyl alcohol (IPA), as well as other olefins in the range of C2 to C5 carbon length.

In preferred embodiments, the DH catalysts according to the invention are stable for the conversion of propylene to IPA at higher temperatures than the acidic resins and strong acid solid catalysts.

In another preferred embodiments, the catalysts of the invention function as DH catalysts for the conversion of propylene to IPA over a wider operational range.

In still another embodiment, the DH catalysts of the invention have good hydrolytic stability.

In yet still another embodiment, the DH catalysts of the invention are regenerable.

Moreover, in other embodiments, the DH catalyst of the invention can be run at low water to propylene ratios (Q ratio) without the co-production of large amounts of IPE for the conversion of propylene to IPA.

It is an object of the invention to provide MMOs that are active for the direct hydration of propylene to produce isopropyl alcohol (IPA) without co-production of large amounts of isopropyl ether (IPE).

It is an object of the invention to provide high-temperature stable ZrO₂-based MMOs operating over a wide variation of operating conditions, having good hydrolytic stability, that produce IPA at low water:propylene ratios and which are regenerable.

These and other objects, features, and advantages will become apparent as reference is made to the following detailed description, preferred embodiments, examples, and appended claims.

### DETAILED DESCRIPTION OF THE INVENTION

According to the invention, mixed metal oxide (MMO) catalysts based on ZrO₂ are made by a process comprising co-precipitation of the metal species from solution, followed by calcination. The MMOs of the invention are useful for the production of isopropyl alcohol (IPA).

The mixed metal oxide of the invention comprises at least two metals, at least one of which is zirconium (Zr) and having at least one other metal selected from transition metals other than Zr.

The mixed metal oxide of the invention is zirconium-based (or “ZrO₂-based”), wherein the term “zirconium-based” means that zirconium metal, regardless of oxidation state, is present in the amount of at least greater than 50 wt %, based on the weight of the metals present in the mixed metal oxide catalyst. It is preferred that zirconium be present in the amount of greater than 50.0 wt % to 99.5 wt %, or 60.0 wt %, to 99.0 wt %, or 70.0 wt % to 98.5 wt %, or 80 wt % to 98.0 wt %, balance the at least one other metal. Useful ranges include from any of the minimum amount specified above to any of the maximum amount specified above, e.g., ranges such as >50 wt % to 98.0 wt % and 80 wt % to 99.0 wt % are also contemplated.

The at least one other metal in the mixed metal oxide of the invention is selected from Groups 3-11 of the Periodic Table, i.e., the transition metals, which includes the Lanthanides and Actinides. More than one metal other than zirconium may be present in the MMO, provided that the one or more metals other than zirconium are co-precipitated along with zirconium.

In an embodiment, the transition metal other than Zr is selected from at least one of Groups 3-9 (again, the transition metals including Lanthanides and Actinides. In another embodiment, the transition metal other than Zr is selected from at least one of Ce, Mn, W, Cu, Mo, Fe, and Cr. In still another embodiment the metal is selected from at least one of the transition metals in Groups 3-9. In yet another embodiment they are selected from Ce, W, Mo, and mixtures thereof. In yet another embodiment the metals used are selected from tungsten, molybdenum, copper, manganese, iron and mixtures thereof.

The mixed metal oxides of the invention are made by co-precipitation of the at least two metals from solution. The metal salts used to co-precipitate with the ZrO can include one, two or more metals. The at least two metals are dissolved in a suitable solvent, such as water, and caused to be simultaneously precipitated, such as by addition of a suitable base. A soluble salt of the metal is preferably used, for example: halides, sulfates, nitrates, and polymetallates such as zirconyl chloride, ammonium metal tungstate, cerium sulfate and the like. More than one different salt containing the same metal may be used. The non-metal counter ion (e.g., halide, sulfate, nitrate, and the like) may be the same or different for the at least two metals and may be independently selected.

The term “soluble salt” would be understood by one of ordinary skill in the art to be a relative term and depends on the solvent used. The exact amount of salt that needs to be dissolved is not particularly important except with respect to the time and effort it takes to obtain a useful amount of the mixed metal, but this is no more than “routine” experimentation by one of ordinary skill in possession of the present disclosure.

The base added to cause co-precipitation is preferably aqueous ammonium hydroxide. Although other bases, for example amines or anilines, may be used to cause co-precipitation, it is preferred that a base having as counter ion a metal such as sodium, calcium, and the like, is not used, to avoid incorporation of a metal other than a transition metal in the final mixed metal oxide of the invention.

After addition of the base, above, adjustment of the pH may be necessary to complete co-precipitation, such as by the addition of small amounts of acid, e.g., sulfuric acid. It is preferred that acids having halide as a counter ion (e.g., HCl), or those based on phosphorus (e.g., H₃PO₄), not be used. One of ordinary skill in the art, in possession of the present disclosure, would be able to perform this procedure without undue experimentation.

Typically the product obtained is a slurry, which may optionally be aged for a period of time of from a few minutes to a few days, preferably 1 to 100 hours, more preferably 12 to 72 hours, still more preferably 24 to 72 hours, optionally in the presence of steam, such as by storage in a steambox. The slurry then may be filtered and dried, such as an elevated temperature such as 80° C.±10° C. (but typically below 100° C.). It is then conveniently calcined at an elevated temperature for several hours, such as in the range of 400 to 1000° C., such as 400 to 900° C., preferably 600 to 850° C., for 30 minutes to 12 hours, such as 1 hour to 12 hours, preferably 2 to 8 hours, in an embodiment 3 to 6 hours, optionally under flowing air or an inert gas such as nitrogen, and then allowed to cool. Again, no more than routine experimentation by the ordinary artisan in possession of the present disclosure is necessary to obtain the mixed metal oxide of the invention. The combined aspects of zirconium quantity (>50 wt %) with respect to co-metal(s), co-precipitation of mixed metals, and calcination to obtain the mixed metal oxide, however, are critical.

The mixed metal oxides according to the invention are useful for the production of isopropyl alcohol (IPA). This may be in a batch process, semi-batch process, or a continuous process.

The DH process according to the invention may use conventional process parameters and/or apparatus for the hydration of the propylene directly to IPA. According to the preferred process of the invention, the hydration of the olefins to alcohols is carried out directly and in a single step, by contacting the olefin with the hydration water with at least one MMO according to the invention. The propylene feed is preferably chemical grade propylene or better. A decrease in the presence of impurities typically maintains catalyst life. The direct hydration process of the invention may be carried out in vapor-phase, liquid-phase or mixed phase.

In one embodiment, the direct hydration process utilizes a fixed bed reactor containing at least one of the MMO catalysts of the invention. The reactor is preferably operated at a pressure ranging from about 200 psig (1379 pKa) to about 2000 psig (13,790 kPa), a temperature ranging from about 80° C. to about 280° C., a water to feed olefin molar ratio (Q ratio) ranging from about 0.1 to about 20 using at least one of the mixed metal oxides as described in this invention. Typically recycle of unconverted olefin is employed to maximize total yields.

In another embodiment, the direct hydration process can utilize a catalytic distillation column for the reaction step and initial distillation. Catalytic distillation per se is well-known. Preferred conditions for this process range from 20 psig to 500 psig, temperatures from about 80° C. to 250° C., and a water to olefin ratio (Q ratio) of 0.1 to 10. The catalyst according to the invention is provided in the distillation column. The feed comprising propylene and water contacts the catalyst and the desired product is recovered, typically as a 99+% product bottoms by simultaneous catalytic hydration and distillation. Unconverted propylene is taken overheads.

One of the advantages of the present invention is that process temperature may include higher temperatures than that commercially acceptable using conventional catalysts. In preferred embodiments, process temperature ranges may be from greater than 140 to 280° C., or greater than 150 to 270° C., or 160 to 260° C. or 170 to 250° C., or 180 to 240° C., or 180 to 220° C., with other ranges contemplated such as from any of the minimum temperatures listed to any of the maximum temperatures listed, e.g., 170 to 240° C.

Further details regarding direct hydration processes may be found in Industrial Organic Chemistry, 2nd Revised and Extended Edition, Section 8.1.2, pp. 194-97 (1993) by K. Weisselmel and H.-J. Arpe, U.S. Pat. No. 7,102,037 and U.S. Patent Application Publication 2006-0281954.

The following examples are meant to illustrate the present invention and provide a comparison with other methods. Numerous modifications and variations are possible and it is to be understood that within the scope of the appended claims, the invention may be practiced otherwise than as specifically described herein.

In the following examples, the amount of metal other than zirconium in the final calcined catalyst is indicated parenthetically, based on wt % of the total metal content (i.e., remainder zirconium wt %).

### Example 1

(2% Ce/Zr) Five hundred grams of ZrOCl₂.8H₂O and 14 grams of Ce(SO₄)₂ were dissolved with stirring in 3.0 liters of distilled water. Another solution containing 260 grams of concentrated NH₄OH and 3.0 liters of distilled water was prepared. These two solutions were combined at the rate of 50 ml/min using a mixing nozzle. The pH of the final composite was adjusted to approximately 8 by the addition of concentrated sulfuric acid. This slurry was then put in polypropylene bottles and placed in a steambox (100° C.) for 72 hours. The product formed was recovered by filtration, washed with excess water, and stored as a filtercake. The filtercake is dried overnight at 85° C. The elemental analyses were Zr—63.7 weight % and Ce—2.92 weight %. Thereafter the filtercake is calcined at 700° C. for a total of 3 hours in flowing air and then allowed to cool.

### Example 2

(6% Ce/Zr) One hundred and twenty-five grams of ZrOCl₂.8H₂O and 14 grams of Ce(SO₄)₂ were dissolved with stirring in 1.5 liter of distilled water. Another solution containing 65 grams of concentrated NH₄OH and 1.5 liters of distilled water was prepared. These two solutions were combined at the rate of 50 ml/min using a mixing nozzle. The pH of the final composite was adjusted to approximately 8 by the addition of concentrated sulfuric acid. This slurry was then put in polypropylene bottles and placed in a steambox (100° C.) for 72 hours. The product formed was recovered by filtration, washed with excess water, and stored as a filtercake. The filtercake is dried overnight at 85° C. The elemental analyses were Zr—58.1 weight % and Ce—9.01 weight %. Thereafter the filtercake is calcined at 700° C. for a total of 3 hours in flowing air and then allowed to cool.

### Example 3

(17% Ce/Zr) One hundred thirty one grams of ZrO(NO₃)₂.xH2O and 28.4 grams of Ce₂(SO₄)₃ were dissolved with stirring in 528 grams of distilled water. A second solution containing 65.9 grams of concentrated NH₄OH and 366.9 grams of distilled water was prepared. These two solutions were combined at the rate of 50 ml/min using a mixing nozzle. The pH of the final composite was adjusted to approximately 8 by the addition of concentrated ammonium hydroxide. This slurry having a solids content of about 7%, was then aged in an autoclave at 100° C. for 72 hours. The product formed was recovered by filtration, washed with excess water, and stored as a filtercake. The filtercake was dried overnight at 120° C. Thereafter, the filtercake was calcined at 700° C. for a total of 3 hours in flowing air and then allowed to cool. The elemental analyses are shown in Table 1.

### Example 4

(20% Ce/Zr) Five hundred grams of ZrOCl₂.8H₂O and 140 grams of Ce(SO₄)₂ were dissolved with stirring in 3.0 liters of distilled water. Another solution containing 260 grams of concentrated NH₄OH and 3.0 liters of distill water was prepared. These two solutions were combined at the rate of 50 ml/min using a mixing nozzle. The pH of the final composite was adjusted to approximately 8 by the addition of concentrated sulfuric acid. This slurry was then put in polypropylene bottles and placed in a steambox (100° C.) for 72 hours. The product formed was recovered by filtration, washed with excess water, and stored as a filtercake. The filtercake is dried overnight at 100° C. The cerium content was analyzed as 17.6%. Thereafter the filtercake is calcined at 700° C. for a total of 3 hours in flowing air and then allowed to cool.

### Example 5

(16% Ce/16% W//Zr) One hundred grams of ZrOCl₂.8H₂O was dissolved with stirring in 300 milliliters of distilled water. Another solution containing 22.4 grams of Ce(SO₄)₂, 10.8 grams of (NH₄)6H₂W₁₂O₄₀.xH₂O and 300 milliliters of distilled water was prepared. These two solutions were combined with stirring. The pH of the final composite was adjusted to approximately 8 by the addition of concentrated NH₄OH. This slurry was then put in polypropylene bottles and placed in a steambox (100° C.) for 72 hours. The product formed was recovered by filtration, washed with excess water, and stored as a filtercake. The filtercake is dried overnight at 85° C. The elemental analyses were Zr—33.6 weight %, W—13.4 weight %, and Ce—13 weight %. Thereafter the filtercake is calcined at 700° C. for a total of 3 hours in flowing air and then allowed to cool.

### Example 6

(16% W/Zr) One thousand grams of ZrOCl₂.8H₂O were dissolved with stirring in 3.0 liters of distilled water. Another solution containing 400 grams of conc. NH₄OH, 108 grams of (NH₄)6H₂W₁₂O₄₀.xH₂O and 3.0 liters of distilled water was prepared. Both solutions were heated to 60° C. These two heated solutions were combined at the rate of 50 ml/min using a mixing nozzle. The pH of the final composite was adjusted to approximately 9 by the addition of concentrated ammonium hydroxide. This slurry was then put in polypropylene bottles and placed in a steambox (100° C.) for 72 hours. The product formed was recovered by filtration, washed with excess water, and dried overnight at 85° C. Elemental analyses were Zr—51.2 weight % and W—21.2 weight %. A sample of this catalyst was calcined to 800° C. in flowing air for 3 hours.

### Example 7

(2% Mn/16% W/Zr) Two hundred and fifty grams of ZrOCl₂.8H₂O were dissolved with stirring in 1.5 liters of distilled water. To this solution was added 5.0 grams of MnSO₄.5H₂O. Another solution containing 130 grams of conc. NH₄OH, 27 grams of (NH₄)6H₂W₁₂O₄₀.xH₂O and 1.5 liters of distilled water was prepared. Both solutions were heated to 60° C. These two heated solutions were combined at the rate of 50 ml/min using a mixing nozzle. The pH of the final composite was adjusted to approximately 9 by the addition of concentrated ammonium hydroxide. This slurry was then put in polypropylene bottles and placed in a steambox (100° C.) for 72 hours. The product formed was recovered by filtration, washed with excess water, and dried overnight at 85° C. Elemental analyses were Zr—52.3 weight %, W—19.3 weight %, and Mn—1.17 weight %. A sample of this catalyst was calcined to 800° C. in flowing air for 3 hours.

### Example 8

(16% Mo/Zr) Five hundred grams of ZrOCl₂.8H₂O were dissolved with stirring in 3.0 liters of distilled water. Another solution containing 260 grams of conc. NH₄OH, 66 grams of (NH₄)₆Mo₇O₂₄.4H₂O and 3.0 liters of distilled water was prepared. Both solutions were heated to 60° C. These two heated solutions were combined at the rate of 50 ml/min using a mixing nozzle. The pH of the final composite was adjusted to approximately 9 by the addition of concentrated ammonium hydroxide. This slurry was then put in polypropylene bottles and placed in a steambox (100° C.) for 72 hours. The product formed was recovered by filtration, washed with excess water, and dried overnight at 85° C. Elemental analyses were Zr—56.1 weight % and Mo—10.5 weight %. A sample of this catalyst was calcined to 800° C. in flowing air for 3 hours.

### Example 9

(1% Cu/16% W/Zr) Five hundred grams of ZrOCl₂.8H₂O were dissolved with stirring in 1.5 liters of distilled water. To this solution was added 6.8 grams of CuSO₄.5H₂O. Another solution containing 260 grams of conc. NH₄OH, 54 grams of (NH₄)6H₂W₁₂O₄₀.xH₂O and 3.0 liters of distilled water was prepared. Both solutions were heated to 60° C. These two heated solutions were combined at the rate of 50 ml/min using a mixing nozzle. The pH of the final composite was adjusted to approximately 9 by the addition of concentrated ammonium hydroxide. This slurry was then put in polypropylene bottles and placed in a steambox (100° C.) for 72 hours. The product formed was recovered by filtration, washed with excess water, and dried overnight at 85° C. Elemental analyses were Zr—49.8 weight %, W—19.1 weight %, and Cu—0.62 weight %. A sample of this catalyst was calcined to 700° C. in flowing air for 3 hours.

### Example 10

(16% W/Zr) One hundred grams of ZrOCl₂.8H₂O was dissolved with stirring in 300 milliliters of distilled water. Another solution containing 10.8 grams of (NH₄)6H₂W₁₂O₄₀.xH₂O and 300 milliliters of distilled water was prepared. These two solutions were combined with stirring. The pH of the final composite was adjusted to approximately 8 by the addition of concentrated NH₄OH. This slurry was then put in polypropylene bottles and placed in a steambox (100° C.) for 72 hours. The product formed was recovered by filtration, washed with excess water, and stored as a filtercake. The filtercake is dried overnight at 85° C. The elemental analyses were Zr—48.3 weight % and W—19.4 weight %. Thereafter the filtercake is calcined at 700° C. for a total of 3 hours in flowing air and then allowed to cool.

The catalysts were tested by running experiments on a continuous pilot plant using a Robinson-Mahoney type CSTR reactor. The catalysts were suspended in a basket surrounding the stirrer that allowed circulated feed to pass through the catalyst. Propylene (93% purity, balance propane) was fed at supercritical conditions and water was injected at moderate molar ratios of 0.2-3.0 to the propylene fed. The ratio of moles of water to moles of propylene is known as the Q ratio and was carefully controlled. Feed to the reactor was introduced from the top and product removed from the bottom. Since the net flow of water was downward across the catalyst, trickle bed conditions were maintained for most of the experiments. This configuration prevented the accumulation of a water layer in the reactor and insured the proper low Q conditions. Stirrer speeds were selected to insure even distribution of the water and propylene feeds across the catalyst. Preferred stirrer speeds are above 2000 rpm.

Catalysts were run under different conditions to discover the trends in hydration performance. In particular, temperature, pressure, Q ratio, and weight hourly space velocity (WHSV) were major variables. Stirrer speed was kept constant for most of the experiments. The following Table 1 demonstrates the effectiveness of the various zirconium oxide MMO catalysts toward propylene direct hydration (DH). All of the catalyst showed some ability for DH. In our experiments the best performing catalysts are the ZrO₂-based catalysts that have been co-precipitated with various transition metals and their oxides. By proper selection of the aforementioned variables and the appropriate MMO according to the invention, in preferred embodiments less than 0.2 mole % by-product IPE is detectable using routine GC equipment. A typical analysis uses a 6890 Agilent GC, a boiling point capillary column (such as a 60 m×0.32 mmID 3 μfilm DB-1), FID detector and integration of the peaks by a standard integration software. However it is to be understood that the particular method of detection of by-product IPE is not critical and any commercially acceptable method may be used, so the “less than 0.2 mole %” will vary within these limitations.

Analysis of the products was by GC, using the method described above. Mass balances were also run to verify the results. Performance was measured by the mole % conversion of propylene and by the selectivity to isopropyl alcohol. All of the MMO catalyst exhibited excellent selectivity to IPA. The results for the ZrO₂-based MMOs are reported in Table 1 below. Several of the MMO's were quite active for DH. Only a few experiments were run for each catalyst and the results in the table do not necessarily reflect the highest possible conversion for that catalyst but only the highest conversion achieved for the experiments performed. It is likely that higher conversions are possible with optimization of the process conditions.

For comparison purposes Amberlyst™ 36 resin was run on this same system. As expected the Amberlyst™ 36 was more active than the MMOs at a lower temperature range of 140° C. to 160° C.; however, as the temperature increased selectivity fell rapidly. In addition, at the higher temperatures, above 140° C., desulfonation is known to deactivate the resin catalyst.

Another example tested was that of pure calcined CeO₂ catalyst that contained no significant amount of ZrO₂ (top example in the table below). That catalyst showed very little activity for DH. When the CeO₂ was combined by coprecipitation with ZrO₂ as in Example 4 it moderated the activity of the ZrO₂.

These results show that the family of MMO's formed by the co-precipitation of ZrO₂ and transition metal oxides from Groups 3-11 form a viable set of catalysts that can be used for direct hydration of C₃═. The higher temperatures and low Q ratios seen for these catalysts extends the available window of operability relative to existing commercialized processes.

Although these experiments were all run with pure water feed it is recognized that in a commercial process recycle of some process water, containing IPA, to the feed may be necessary. It is expected that the recycled IPA will improve the solubility of C₃═ into the aqueous phase. The addition of IPA, or another miscibility agent, can be used to enhance the reactivity of the system and potentially improve the economics of a commercial process.

TABLE 1

| | C3 | H2O | | Flow | | | Measured | Measured |
|---|---|---|---|---|---|---|---|---|
| | Feed | Feed | | Rate | | | C3 = | IPA |
| Example # - | flow, | flow, | Q | (WHSV) | Temp | Pressure | Conversion | Selectivity |
| Catalyst | cc/hr | cc/hr | ratio | hr − 1 | ° C. | psig | mole % | mole % |
| 68%CeO2 | 10 | 2 | 1.0 | 0.6 | 220 | 1000 | 0.6% | 100.0% |
| Ex 4 - 20%CeZrO2 | 10 | 2 | 1.0 | 1.8 | 230 | 1000 | 1.0% | 100.0% |
| Ex 5 - | 10 | 2 | 1.0 | 1.7 | 220 | 1000 | 1.5% | 100.0% |
| 16%Ce16%WZrO2 | | | | | | | | |
| Ex 2 - 6%CeZrO2 | 15 | 3 | 1.0 | 1.7 | 230 | 1700 | 2.5% | 100.0% |
| Ex 3 - 17%CeZrO2 | 10 | 2 | 1.0 | 1.1 | 250 | 1700 | 4.7% | 100.0% |
| Ex 1 - 2%CeZrO2 | 10 | 0.8 | 0.4 | 1.0 | 220 | 1000 | 6.4% | 92.5% |
| Ex 7 - | 10 | 2 | 1.0 | 1.0 | 200 | 1000 | 7.9% | 99.1% |
| 2%Mn16%WZrO2 | | | | | | | | |
| Ex 9 - | 10 | 2 | 1.0 | 1.0 | 200 | 1000 | 11.7% | 97.9% |
| 1%Cu16%WZrO2 | | | | | | | | |
| Ex 8 - | 10 | 2 | 1.0 | 0.8 | 180 | 1000 | 12.5% | 96.0% |
| 16%MoZrO2 | | | | | | | | |
| Ex 6 - 16%WZrO2 | 10 | 3 | 1.5 | 1.5 | 180 | 1000 | 15.6% | 97.0% |
| Ex 10 - 16%WZrO2 | 10 | 2 | 1.0 | 1.0 | 200 | 1000 | 17.9% | 94.2% |
| Amberlyst ™ 36 | 10 | 2 | 1.0 | 1.5 | 140 | 1000 | 15.0% | 94.7% |
| Amberlyst ™ 36 | 10 | 2 | 1.0 | 1.5 | 160 | 1000 | 58.4% | 81.2% |

The MMOs of the invention may also be used to produce other C2 to C5 alcohols by the direct hydration method and in embodiments includes the production of a mixture of such alcohols using a feedstream comprising a mixture of C2 to C5 olefins, as well as the production of individual C2 to C5 alcohols using a feedstream consisting essentially of the appropriate individual alcohol.

While the illustrative embodiments of the invention have been described with particularity, it will be understood that various other modifications will be apparent to and can be readily made by those skilled in the art without departing from the spirit and scope of the invention. Nevertheless preferred embodiments may be specified as the following: (1) a mixed metal oxide comprising more than 50 wt % Zr, based on the weight of the metals in said oxide, and at least one other metal, other than Zr, selected from Groups 3-11 of the Periodic Table, made by a process comprising: (a) co-precipitation from solution of Zr and said at least one other metal to obtain a co-precipitate, and then (b) calcining said co-precipitate to obtain said mixed metal oxide; (2) the mixed metal oxide as described in (1); (3) a method of making one or more C2-C5 alcohols, preferably including IPA alone or together with at least one other C2-C5 alcohol, by direct hydration of the appropriate C2-C5 olefins (propylene in the case of IPA) comprising contacting a feedstream comprising the appropriate olefin (e.g., propylene in the case of IPA) and water with a catalyst according to (1) or (2). It will be recognized by the ordinarily skilled artisan reading the present disclosure that the method of making the MMO of the invention and/or the method of using it may be preferably modified by one or more of the following: (i) wherein said co-precipitation is initiated by addition of basic solution, particularly by addition of ammonium hydroxide, and optionally wherein the addition of said basic solution (preferably NH₄OH) is followed by the addition of an acid, preferably sulfuric acid, and preferably so as to adjust the pH of the solution in the range of about 7.5-9.5, more preferably in the range of about 8.0-9.0; and/or (ii) wherein there is a step between steps (a) and (b) of aging the filter cake of said co-precipitate (such as obtained by filtration) in a steam box for from 1 to 100 hours; and/or (iii) wherein the calcining comprises heating said co-precipitate (preferably after filtering and aging) at a temperature of between about 400° C. and 1000° C. (such as 400 to 900° C., preferably 600-850° C., such as 600-750° C.) for a period of about 30 minutes to about 12 hours or 2-8 hours; (iv) and/or the preferred embodiment wherein the at least one other metal is selected from Ce, Mn, W, Cu, Mo, Fe, Cr, and mixtures thereof, or at least one of the metals from Groups 3-9 of the Periodic Table; and/or the more preferred embodiment wherein Zr is provided by ZrOCl₂; (v) and/or the more preferred embodiment wherein the at least one other metal is provided to step (a) as the sulfate salt. The most preferred embodiment of the invention, however, is the method of making IPA by direct hydration of propylene comprising contacting a feedstream comprising propylene and water with a catalyst as described herein, particularly in this paragraph, the catalyst preferably being a mixed metal oxide comprising more than 50 wt % Zr, based on the weight of the metals in said oxide, and at least one other metal, other than Zr, selected from Groups 3-11 of the Periodic Table, made by a process comprising: (a) co-precipitation from solution of Zr and said at least one other metal to obtain a co-precipitate, and then (b) calcining said co-precipitate to obtain said mixed metal oxide, still more preferably modified by one or more of the following: (i) wherein said co-precipitation is initiated by addition of basic solution, particularly by addition of ammonium hydroxide, and optionally wherein the addition of said basic solution (preferably NH₄OH) is followed by the addition of an acid, preferably sulfuric acid, and preferably so as to adjust the pH of the solution in the range of about 7.5-9.5, more preferably in the range of about 8.0-9.0; and/or (ii) wherein there is a step between steps (a) and (b) of aging the filter cake of said co-precipitate (such as obtained by filtration) in a steam box for from 1 to 100 hours; and/or (iii) wherein the calcining comprises heating said co-precipitate (preferably after filtering and aging) at a temperature of between about 400° C. and 1000° C. (such as 400 to 900° C., preferably 600-850° C., such as 600-750° C.) for a period of about 30 minutes to about 12 hours, or 1 hour to 12 hours, or 2-8 hours; (iv) and/or the preferred embodiment wherein the at least one other metal is selected from Ce, Mn, W, Cu, Mo, Fe, Cr, and mixtures thereof, or at least one of the metals from Groups 3-9 of the Periodic Table; and/or the more preferred embodiment wherein Zr is provided by ZrOCl₂; (v) and/or the more preferred embodiment wherein the at least one other metal is provided to step (a) as the sulfate salt; and/or in embodiments, wherein the process is further characterized (or solely characterized) by at least one of the following: (i) wherein IPA is produced with at least 95% selectivity (or 98% selectivity, or 99% selectivity, or wherein IPE cannot be detected by routine GC analysis); (ii) wherein the Q ratio is optimized in the range of 0.2 to 20 to selectively produce IPA without IPE by-product, as measure by GC; (iii) wherein said feedstream is provided to a catalytic distillation column packed with said catalyst and IPA is recovered as overheads; (iv) wherein said feedstream further comprises at least one C2 to C5 olefin other than propylene and wherein said at least one C2 to C5 olefin other than propylene is directly hydrated to the corresponding alcohol; (v) further comprising recycle of at least a portion of at least one of (a) process water and (b) product IPA; (vi) wherein said direct hydration occurs at a temperature of from greater than 160 to about 250° C., or from about 180 to about 240° C., or from about 180 to about 220° C.; the process of wherein said feedstream further comprises a miscibility agent, such as IPA; the fixed bed process wherein in embodiments the direct hydration occurs at a pressure of from about 200 to 2000 psi; and the process using catalytic distillation wherein in embodiments said process occurs at a pressure of from about 20 to 500 psi and in other embodiments from about 20 to 300 psi.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated.

What is claimed is:

1-15. (canceled)

16. A mixed metal oxide comprising more than 50 wt % Zr, based on the weight of the metals in said oxide, and at least one other metal, other than Zr, selected from Groups 3-11 of the Periodic Table, made by a process comprising
(a) co-precipitation from solution of Zr and said at least one other metal to obtain a co-precipitate, and then
(b) calcining said co-precipitate to obtain said mixed metal oxide.

17. The mixed metal oxide according to claim 16, wherein said co-precipitation is initiated by addition of basic solution to said solution of Zr and other metal.

18. The mixed metal oxide according to claim 16, wherein said co-precipitation is initiated by addition of ammonium hydroxide to said solution of Zr and other metal.

19. The mixed metal oxide according to claim 16, wherein said co-precipitation is initiated by addition of ammonium hydroxide to said solution of Zr and other metal, and optionally completed by addition of sulfuric acid, to adjust the final pH of the resulting solution to from about 7.5 to about 9.5.

20. The mixed metal oxide according to claim 16, wherein said process further includes a step between steps (a) and (b) of aging a filter cake of said co-precipitate in a steam box for from 1 to 100 hours.

21. The mixed metal oxide according to claim 16, wherein said calcining comprises heating said co-precipitate at a temperature of between about 400° C. and 1000° C. for a period of about 30 minutes to about 12 hours.

22. The mixed metal oxide according to claim 16, wherein said calcining comprises heating said co-precipitate at a temperature of about 600° C. to 850° C. for a period of about 2 to about 8 hours.

23. The mixed metal oxide according to claim 16, wherein said at least one other metal is selected from Ce, Mn, W, Cu, Mo, Fe, Cr, and mixtures thereof.

24. The mixed metal oxide according to claim 16, wherein said at least one other metal is selected from at least one of Groups 3-9 of the Periodic Table.

25. The mixed metal oxide according to claim 16, wherein Zr is provided by ZrOCl₂.

26. The mixed metal oxide according to claim 1, wherein the at least one other metal is provided to step (a) as a sulfate salt.

27. A method of making isopropylalcohol (IPA) by direct hydration of propylene comprising contacting a feedstream comprising propylene and water with a catalyst comprising a mixed metal oxide according to claim 1.

28. The method of claim 27, wherein IPA is produced with at least 95% selectivity.

29. The method of claim 27, wherein IPA is produced with at least 98% selectivity.

30. The method of claim 27, wherein the molar ratio of water to propylene in the feedstream (Q ratio) is optimized in the range of 0.2 to 20 to selectively produce IPA with less than 0.2 mole % isopropylether (IPE) by-product, as measured by gas chromatography (GC).

31. The method of claim 27, wherein said feedstream further comprises at least one C2 to C5 olefin other than propylene and wherein said at least one C2 to C5 olefin other than propylene is directly hydrated to the corresponding alcohol.

32. The method of claim 27, further comprising recycle of at least a portion of at least one of (a) process water and (b) product IPA.

33. The method of claim 27, wherein said direct hydration occurs at a temperature of from greater than 160 to about 250° C.

34. The process of claim 27, wherein said direct hydration is a fixed bed direct hydration at a pressure of from about 200 to 2000 psig and at a temperature of from about 180 to about 240° C., and further wherein IPA is added to the feedstream.

35. The process of claim 27, wherein said feedstream is provided to a catalytic distillation column packed with said catalyst and IPA is recovered as bottoms product.
